(19) United States
(12) Patent Application Publication
(10) Pub. No.: US 2026/0151148 A1
(43) Pub. Date: Jun. 4, 2026

(54) METHOD FOR TREATING CONDITIONS OF THE GI TRACT USING A VIBRATING INGESTIBLE CAPSULE

(71) Applicant: Vibrant Ltd., Yokneam (IL)

(72) Inventors: Lior BEN-TSUR, Netanya (IL); Camille MORLIERE, Adelaide (AU)

(21) Appl. No.: 19/464,442

(22) Filed: Jan. 29, 2026

Related U.S. Application Data
(63) Continuation of application No. 17/117,822, filed on Dec. 10, 2020.
Application No. 17/117,822 is a continuation-in-part of application No. 15/882,283, filed on Jan. 29, 2018, now abandoned.
Application No. 17/117,822 is a continuation-in-part of application No. 16/823,035, filed on Mar. 18, 2020, now abandoned.
Application No. 16/823,035 is a continuation-in-part of application No. 15/882,536, filed on Jan. 29, 2018, now abandoned.
(60) Provisional application No. 62/451,827, filed on Jan. 30, 2017.
(60) Provisional application No. 62/451,831, filed on Jan. 30, 2017.

(30) Foreign Application Priority Data
Mar. 30, 2018 (GB) 1805325.6
May 31, 2018 (GB) 1808859.1

(51) Int. Cl. A61B 17/22; A61B 17/00; A61H 23/02

(52) U.S. Cl. CPC A61B 17/22012; A61H 23/02; A61B 2017/00075; A61B 2017/22037; A61H 2205/083; A61H 2230/00

(57) ABSTRACT

A method for treating conditions of the gastrointestinal tract of a subject using a vibrating ingestible capsule ingested by the subject and activated in a targeted zone of the gastrointestinal tract of the subject. The conditions may include a sensation of gastric bloating, a sensation of straining during defecating, diarrhea and/or gastroparesis. The method is further suitable for reducing Bristol stool scores of fecal matter of the subject and for increasing a Bristol stool score of fecal matter of the subject.

### RELATED APPLICATIONS

U.S. patent application Ser. No. 17/117,822, filed on Dec. 10, 2020 is incorporated herein by reference in its entirety. U.S. patent application Ser. No. 17/117,822 is a Continuation in Part of U.S. patent application Ser. No. 15/882,283 filed Jan. 29, 2018, which gains priority from U.S. Provisional Patent Application No. 62/451,827 filed Jan. 30, 2017, both entitled METHOD FOR TREATING A GASTRIC BLOATING SENSATION USING A VIBRATING INGESTIBLE CAPSULE. The present application is also a Continuation in Part of U.S. patent application Ser. No. 15/882,289 filed Jan. 29, 2018, which gains priority from U.S. Provisional Patent Application No. 62/451,834 filed Jan. 30, 2017, both entitled METHOD FOR TREATING DIARRHEA AND REDUCING BRISTOL STOOL SCORES USING A VIBRATING INGESTIBLE CAPSULE. The present application is further a Continuation in Part of U.S. patent application Ser. No. 15/882,329 filed Jan. 29, 2018, which gains priority from U.S. Provisional Patent Application No. 62/451,837 filed Jan. 30, 2017, both entitled METHOD FOR TREATING GASTROPARESIS USING A VIBRATING INGESTIBLE CAPSULE. The present application is also a continuation in part of U.S. patent application Ser. No. 15/882,536, filed Jan. 29, 2018, which gains priority from U.S. Provisional Patent Application No. 62/451,831 filed Jan. 30, 2017, both entitled METHOD FOR REDUCING A STRAINING SENSATION DURING DEFECATING AND IMPROVING BRISTOL STOOL SCORES USING A VIBRATING INGESTIBLE CAPSULE.

U.S. patent application Ser. Nos. 15/882,283; 15/882,289; 15/882,536; and Ser. No. 15/882,329, as well as U.S. Provisional Patent Application Nos. 62/451,827; 62/451,834; 62/451,831; and 62/451,837 are each incorporated herein by reference as if fully set forth herein.

### FIELD OF THE INVENTION

The present invention relates in general to methods of treating conditions of the gastrointestinal tract using a vibrating ingestible capsule. Specifically, the present invention relates to methods of treating a gastric bloating sensation within the stomach or midriff region of a human subject using a vibrating ingestible capsule. The present invention relates to methods of treating diarrhea and reducing Bristol stool scores using a vibrating ingestible capsule. The present invention relates to methods of treating gastroparesis using a vibrating ingestible capsule. The present invention relates to methods for reducing a straining sensation of a human subject during defecating and increasing Bristol stool scores of fecal matter of the subject using a vibrating ingestible capsule.

### SUMMARY OF THE INVENTION

In accordance with an embodiment of the present invention, there is provided a method of treating two opposite sensations within a stomach or midriff region of a human subject, or two opposite conditions of the gastrointestinal tract of the human subject, the method including:
(a) providing, to the human subject suffering from one of the two opposite sensations or one of the two opposite conditions, a vibrating gastrointestinal capsule adapted to transit an alimentary canal of a human, the vibrating gastrointestinal capsule having:
   a housing;
   a battery, disposed within the housing; and
   a vibrating agitator, powered by the battery, the vibrating agitator adapted such that, in a first vibrating mode of operation, the housing exerts vibrations on an environment surrounding the vibrating gastrointestinal capsule, the vibrating gastrointestinal capsule being controllable to effect the first vibrating mode of operation; and
(b) in response to the human subject feeling one of the two opposite sensations or suffering from one of the two opposite conditions, ingesting, by the human subject, the vibrating gastrointestinal capsule, to treat the one of the two opposite sensations or the one of the two opposite conditions,
wherein a frequency of vibration employed by the vibrating ingestible capsule during treatment of the two opposite conditions or the two opposite sensations, within the gastrointestinal tract of the human subject, is in the range of 100 Hz to 650 Hz, regardless of which of the two opposite conditions or the two opposite sensations is experienced by the human subject.

In some embodiments, a targeted zone at which vibration is applied by the vibrating ingestible capsule during treatment of the two opposite conditions or the two opposite sensations is the same regardless of which of the two opposite conditions or the two opposite sensations is experienced by the human subject.

In some embodiments, the two opposite conditions include diarrhea and constipation, and wherein the vibrating gastrointestinal capsule is adapted to treat diarrhea and constipation using the same treatment protocol.

In some embodiments, a first condition of the two opposite conditions is manifested by hard stool defecation and a second condition of the two opposite conditions is manifested by soft stool defecation, wherein the hard stool defecation is characterized by a stool having a first Bristol stool score lower than 2 and the soft stool defecation is characterized by a stool having a second Bristol stool score higher than 5, and wherein the vibrating gastrointestinal capsule is adapted to increase the first Bristol stool score and to decrease the second Bristol stool score using the same treatment protocol.

In accordance with an embodiment of the present invention, there is provided a method of treating a sensation of gastric bloating within a stomach or midriff region of a human subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that at least a portion of the first vibrating mode of operation occurs when the capsule is disposed within a targeted zone within a gastrointestinal tract of the subject, so as to alleviate or dissipate the sensation of gastric bloating.

In some embodiments, the targeted zone includes an intestinal section of the gastrointestinal tract of the subject.

In some embodiments, the targeted zone is in the stomach of the subject.

In some embodiments, controlling includes pre-setting an activation time delay of the capsule, prior to the ingesting.

In some embodiments, the targeted zone includes an intestinal section of the gastrointestinal tract of the subject, and the activation time delay is in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, the targeted zone is in the stomach of the subject, and the activation time delay is in the range of 1 minute to 6 hours, 1 minute to 5 hours, 1 minute to 4 hours, 1 minute to 3 hours, 1 minute to 2 hours, 5 minutes to 6 hours, 5 minutes to 5 hours, 5 minutes to 4 hours, 5 minutes to 3 hours, 5 minutes to 2 hours, 10 minutes to 6 hours, 10 minutes to 5 hours, 10 minutes to 4 hours, 10 minutes to 3 hours, or 10 minutes to 2 hours.

In some embodiments, the subject is a particular subject and pre-setting of the activation time delay is according to a transit time of chyme along the gastrointestinal tract of the particular subject.

In some embodiments, the method further includes, prior to pre-setting of the activation time delay, obtaining information relating to a transit time of chime along the gastrointestinal tract of the particular subject.

In some embodiments, the vibrating agitator includes at least a radial agitation mechanism adapted, in the first vibrating mode of operation, to exert radial forces on the housing, in a radial direction with respect to a longitudinal axis of the housing, thereby to cause the vibrations of the housing. In some embodiments, the radial agitation mechanism includes unbalanced weight attached to a shaft of an electric motor powered by the battery.

In some embodiments, the vibrating agitator includes at least an axial agitation mechanism adapted, in the first vibrating mode of operation, to exert axial forces on the housing, in an axial direction with respect to a longitudinal axis of the housing, thereby to cause the vibrations of the housing. In some embodiments, the axial agitation mechanism includes an electric motor powered by the battery and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitator is adapted in the first vibrating mode of operation, to exert radial forces on the housing in a radial direction with respect to the longitudinal axis of the housing and to exert axial forces on the housing in an axial direction with respect to the longitudinal axis of the housing, thereby to cause the vibrations of the housing. In some embodiments, the vibrating agitator includes a radial agitation mechanism adapted to exert the radial forces and a separate axial agitation mechanism adapted to exert the axial forces. In other embodiments, the vibrating agitator includes a single agitation mechanism adapted to exert the radial forces and the axial forces.

In some embodiments, the vibrating mode of operation including a plurality of cycles, each of the cycles including a vibration duration followed by a repose duration, wherein the housing exerts the vibrations during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 12 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitator is configured such that a net force exerted by the housing on the environment is in the range of 50 grams-force to 600 grams-force.

In some embodiments, the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitator is effected so as to effect a mechanical stimulation of the wall of the gastrointestinal tract in the targeted zone.

In some embodiments, the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

In some embodiments, the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

In some embodiments, the subject is a constipation free subject.

In some embodiments, the subject is a subject diagnosed with irritable bowel syndrome. In some embodiments, the subject is a subject suffering from at least one food allergy or food intolerance. In some embodiment, the subject is a subject suffering from enzymatic deficiency. In some embodiments, the subject is a subject suffering from hormonal deficiency. In some embodiments, the subject is a subject suffering from hormonal imbalance.

In some embodiments, ingesting and controlling together form a treatment session, and wherein the method includes administering to the subject at least one the treatment session.

In some embodiments, administering to the subject at least one treatment session includes administering to the subject a plurality of treatment sessions.

In some embodiments, administering a plurality of treatment sessions includes administering at least one the treatment session per week, over a treatment period of at least two weeks, at least at least three weeks, at least four weeks, at least five weeks, at least six weeks, or at least eight weeks.

In some embodiments, administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

In accordance with an embodiment of the present invention, there is provided a method of treating diarrhea in a human subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that at least a portion of the first vibrating mode of operation occurs when the capsule is disposed within a targeted zone within a gastrointestinal tract of the subject, so as to treat, reduce, or alleviate diarrhea in the subject.

In accordance with another embodiment of the present invention, there is provided a method of reducing a Bristol stool score of fecal matter defecated by a human subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that the first vibrating mode of operation occurs when the capsule is disposed within a targeted zone within a gastrointestinal tract of the subject, so as to reduce the Bristol stool score of fecal matter defecated by the subject.

In some embodiments, the targeted zone includes an intestinal section of the gastrointestinal tract of the subject. In some embodiments, the targeted zone is the stomach of the subject.

In some embodiments, controlling includes pre-setting an activation time delay of the capsule, prior to the ingesting.

In some embodiments, the targeted zone includes an intestinal section of the gastrointestinal tract of the subject, and the activation time delay is in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, the targeted zone is the stomach of the subject, and the activation time delay is in the range of 1 minute to 6 hours, 1 minute to 5 hours, 1 minute to 4 hours, 1 minute to 3 hours, 1 minute to 2 hours, 5 minutes to 6 hours, 5 minutes to 5 hours, 5 minutes to 4 hours, 5 minutes to 3 hours, 5 minutes to 2 hours, 10 minutes to 6 hours, 10 minutes to 5 hours, 10 minutes to 4 hours, 10 minutes to 3 hours, or 10 minutes to 2 hours.

In some embodiments, the subject is a particular subject and wherein the pre-setting of the activation time delay is according to a measured or estimated transit time of chyme along the gastrointestinal tract of the particular subject.

In some embodiments, the method further includes, prior to pre-setting the activation time delay, obtaining information relating to the measured or estimated transit time of chyme along the gastrointestinal tract of the particular subject.

In some embodiments, the vibrating agitator includes at least a radial agitation mechanism adapted, in the first vibrating mode of operation, to exert radial forces on the housing, in a radial direction with respect to a longitudinal axis of the housing, thereby to cause the vibrations of the housing. In some embodiments, the radial agitation mechanism includes unbalanced weight attached to a shaft of an electric motor powered by the battery.

In some embodiments, the vibrating agitator includes at least an axial agitation mechanism adapted, in the first vibrating mode of operation, to exert axial forces on the housing, in an axial direction with respect to a longitudinal axis of the housing, thereby to cause the vibrations of the housing. In some embodiments, the axial agitation mechanism includes an electric motor powered by the battery and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitator is adapted in the first vibrating mode of operation, to exert radial forces on the housing in a radial direction with respect to a longitudinal axis of the housing and to exert axial forces on the housing in an axial direction with respect to the longitudinal axis of the housing, thereby to cause the vibrations of the housing. In some embodiments, the vibrating agitator includes a radial agitation mechanism adapted to exert the radial forces and a separate axial agitation mechanism adapted to exert the axial forces. In some other embodiments, the vibrating agitator includes a single agitation mechanism adapted to exert the radial forces and the axial forces.

In some embodiments, the vibrating mode of operation including a plurality of cycles, each of the cycles including a vibration duration followed by a repose duration, wherein the housing exerts the vibrations during the vibration duration. In some embodiments, the repose duration is greater than the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitator is configured such that a net force exerted by the housing on the environment is in the range of 50 grams-force to 600 grams-force.

In some embodiments, the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitator is effected so as to effect a mechanical stimulation of the wall of the gastrointestinal tract in the targeted zone.

In some embodiments, the subject is a subject who has experienced at least three loose bowel movements daily for at least two weeks preceding treatment.

In some embodiments, the subject is a subject who has experienced at least one loose bowel movement daily for at least one week preceding treatment.

In some embodiments, the subject is a subject whose bowel movements have a Bristol stool score of at least 5.

In some embodiments, ingesting and controlling together form a treatment session, and wherein the method includes administering to the subject at least one the treatment session.

In some embodiments, administering to the subject at least one treatment session includes administering to the subject a plurality of treatment sessions.

In some embodiments, administering a plurality of treatment sessions includes administering at least one the treatment session per week, over a treatment period of at least two weeks, at least at least three weeks, at least four weeks, at least five weeks, at least six weeks, or at least eight weeks.

In some embodiments, administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

In accordance with an embodiment of the present invention, there is provided a method of treating gastroparesis in a human subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that at least a portion of the first vibrating mode of operation occurs when the capsule is disposed within a targeted zone within a gastrointestinal tract of the subject, so as to treat, reduce, or alleviate gastroparesis in the subject.

In some embodiments, the targeted zone includes a section of the gastrointestinal tract of the subject adjacent the pyloric sphincter.

In some embodiments, the targeted zone is in the stomach of the subject.

In some embodiments, the targeted zone is the duodenum of the subject.

In some embodiments, controlling includes pre-setting an activation time delay of the capsule, prior to the ingesting.

In some embodiments, the activation time delay is in the range of 1 second to 5 hours, 1 second to 4 hours, 1 second to 3 hours, 1 second to 2 hours, 1 second to 1 hour, 1 second to 40 minutes, 1 second to 30 minutes, 1 second to 20 minutes, 1 minute to 6 hours, 1 minute to 5 hours, 1 minute to 4 hours, 1 minute to 3 hours, 1 minute to 2 hours, 1 minute to 1 hour, 1 minute to 40 minutes, 1 minute to 30 minutes, 1 minute to 20 minutes, 5 minutes to 6 hours, 5 minutes to 5 hours, 5 minutes to 4 hours, 5 minutes to 3 hours, 5 minutes to 2 hours, 10 minutes to 6 hours, 10 minutes to 5 hours, 10 minutes to 4 hours, 10 minutes to 3 hours, or 10 minutes to 2 hours.

In some embodiments, the subject is a particular subject and wherein the pre-setting of the activation time delay is according to a measured or estimated transit time of chyme along the gastrointestinal tract of the particular subject. In some embodiments, the method further includes, prior to pre-setting the activation time delay, obtaining information relating to the measured or estimated transit time of chyme along the gastrointestinal tract of the particular subject.

In some embodiments, the vibrating agitator includes at least a radial agitation mechanism adapted, in the first vibrating mode of operation, to exert radial forces on the housing, in a radial direction with respect to a longitudinal axis of the housing, thereby to cause the vibrations of the housing. In some embodiments, the radial agitation mechanism includes unbalanced weight attached to a shaft of an electric motor powered by the battery.

In some embodiments, the vibrating agitator includes at least an axial agitation mechanism adapted, in the first vibrating mode of operation, to exert axial forces on the housing, in an axial direction with respect to a longitudinal axis of the housing, thereby to cause the vibrations of the housing. In some embodiments, the axial agitation mechanism includes an electric motor powered by the battery and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitator is adapted in the first vibrating mode of operation, to exert radial forces on the housing in a radial direction with respect to a longitudinal axis of the housing and to exert axial forces on the housing in an axial direction with respect to the longitudinal axis of the housing, thereby to cause the vibrations of the housing.

In some embodiments, the vibrating agitator includes a radial agitation mechanism adapted to exert the radial forces and a separate axial agitation mechanism adapted to exert the axial forces.

In some embodiments, the vibrating agitator includes a single agitation mechanism adapted to exert the radial forces and the axial forces.

In some embodiments, the vibrating mode of operation including a plurality of cycles, each of the cycles including a vibration duration followed by a repose duration, wherein the housing exerts the vibrations during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitator is configured such that a net force exerted by the housing on the environment is in the range of 50 grams-force to 600 grams-force.

In some embodiments, the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitator is effected so as to effect a mechanical stimulation of the wall of the gastrointestinal tract in the targeted zone.

In some embodiments, the subject is a subject who has experienced nausea at least 25% of the time for at least two weeks preceding treatment.

In some embodiments, the subject is a subject who has experienced vomiting after at least one meal per day or after 25% of the meals for at least two weeks preceding treatment.

In some embodiments, the subject is a subject who has experienced a feeling of fullness after eating ten bites or fewer in at least 50% of the meals for at least two weeks preceding treatment.

In some embodiments, the subject is a subject who has experienced heart burn or acid reflux at least 25% of the time for at least two weeks preceding treatment.

In some embodiments, ingesting and controlling together form a treatment session, and wherein the method includes administering to the subject at least one the treatment session.

In some embodiments, administering to the subject at least one treatment session includes administering to the subject a plurality of treatment sessions.

In some embodiments, administering a plurality of treatment sessions includes administering at least one the treatment session per week, over a treatment period of at least two weeks, at least at least three weeks, at least four weeks, at least five weeks, at least six weeks, or at least eight weeks.

In some embodiments, administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

In accordance with an embodiment of the present invention, there is provided a method of treating a sensation of straining while defecating in a human subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that at least a portion of the first vibrating mode of operation occurs when the capsule is disposed within a targeted zone within a gastrointestinal tract of the subject, so as to alleviate or dissipate the sensation of straining while defecating.

In accordance with another embodiment of the present invention, there is provided a method of increasing a Bristol stool score of fecal matter defecated by a subject using a gastrointestinal capsule adapted to transit an alimentary canal of the subject, the capsule having:
a housing;
a battery, disposed within the housing; and
a vibrating agitator, powered by the battery, the vibrating agitator adapted such that, in a first vibrating mode of operation, the housing exerts vibrations on an environment surrounding the capsule, the method including:
   (a) ingesting the gastrointestinal capsule; and
   (b) controlling the vibrating agitator such that the first vibrating mode of operation occurs when the capsule is disposed within a targeted zone within a gastrointestinal tract of the subject, so as to increase the Bristol stool score of fecal matter defecated by the subject.

In some embodiments, the targeted zone includes an intestinal section of the gastrointestinal tract of the subject. In some embodiments, the targeted zone includes a section of a large intestine of the subject. In some embodiments, the targeted zone includes a rectal section of the gastrointestinal tract of the subject.

In some embodiments, controlling includes pre-setting an activation time delay of the capsule, prior to ingesting. In some embodiments, the activation time delay is in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In some embodiments, the subject is a particular subject and pre-setting of the activation time delay is according to a measured or estimated transit time of chyme along the gastrointestinal tract of the particular subject.

In some embodiments the method further includes, prior to pre-setting of the activation time delay, obtaining information relating to the measured or estimated transit time of chime along the gastrointestinal tract of the particular subject.

In some embodiments, the vibrating agitator includes at least a radial agitation mechanism adapted, in the first vibrating mode of operation, to exert radial forces on the housing, in a radial direction with respect to a longitudinal axis of the housing, thereby to cause the vibrations of the housing. In some embodiments, the radial agitation mechanism includes unbalanced weight attached to a shaft of an electric motor powered by the battery.

In some embodiments, the vibrating agitator includes at least an axial agitation mechanism adapted, in the first vibrating mode of operation, to exert axial forces on the housing, in an axial direction with respect to a longitudinal axis of the housing, thereby to cause the vibrations of the housing. In some embodiments, the axial agitation mechanism includes an electric motor powered by the battery and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitator is adapted in the first vibrating mode of operation, to exert radial forces on the housing in a radial direction with respect to a longitudinal axis of the housing and to exert axial forces on the housing in an axial direction with respect to the longitudinal axis of the housing, thereby to cause the vibrations of the housing. In some embodiments, the vibrating agitator includes a radial agitation mechanism adapted to exert the radial forces and a separate axial agitation mechanism adapted to exert the axial forces. In some other embodiments, the vibrating agitator includes a single agitation mechanism adapted to exert the radial forces and the axial forces.

In some embodiments, the vibrating mode of operation including a plurality of cycles, each of the cycles including a vibration duration followed by a repose duration, wherein the housing exerts the vibrations during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitator is configured such that a net force exerted by the housing on the environment is in the range of 50 grams-force to 600 grams-force.

In some embodiments, the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitator is effected so as to effect a mechanical stimulation of the wall of the gastrointestinal tract in the targeted zone.

In some embodiments, the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

In some embodiments, the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

In some embodiments, the subject is a constipation free subject.

In some embodiments, the subject suffers from emotional stress, or has psychosomatically induced symptoms.

In some embodiments, the ingesting and controlling together form a treatment session, and wherein the method includes administering to the subject at least one the treatment session.

In some embodiments, administering to the subject at least one treatment session includes administering to the subject a plurality of treatment sessions.

In some embodiments, administering a plurality of treatment sessions includes administering at least one the treatment session per week, over a treatment period of at least two weeks, at least at least three weeks, at least four weeks, at least five weeks, at least six weeks, or at least eight weeks.

In some embodiments, administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

### BRIEF DESCRIPTION OF THE FIGURES

The foregoing discussion will be understood more readily from the following detailed description of the invention, when taken in conjunction with the accompanying FIGS. 1-8), in which:

FIG. 1 is a schematic block diagram of an vibrating ingestible capsule for treating a condition of the gastrointestinal tract of a human subject according to an embodiment of the present invention;

FIG. 2 is a schematic flowchart of a method for treating a condition of the gastrointestinal tract of a human subject according to the present invention, the treatment being based one use of an ingestible vibrating gastrointestinal capsule;

FIG. 3 is a graphic representation of reduction in a gastric bloating sensation in subjects treated by the method of FIG. 2;

FIG. 4 is a graphic representation of reduction in a gastric bloating sensation in subjects treated by the method of FIG. 2 as compared to subjects treated with a sham capsule;

FIG. 5 is a graphic representation of reduction in a straining sensation in subjects treated by the method of FIG. 2;

FIG. 6 is a graphic representation of a Bristol stool score of fecal matter of subjects treated by the method of FIG. 2;

FIG. 7 is a graphic representation of reduction in a straining sensation in subjects treated by the method of FIG. 2 as compared to subjects treated with a sham capsule; and

FIG. 8 is a graphic representation of a Bristol stool score of fecal matter of subjects treated by the method of FIG. 2 as compared to subjects treated with a sham capsule.

### DESCRIPTION OF THE PREFERRED EMBODIMENTS

The principles of the inventive methods of treating conditions of the gastrointestinal tract of a human subject using a vibrating ingestible capsule, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

We have discovered a method for treating a gastric bloating sensation within a stomach or midriff region of a human using an ingestible vibrating gastrointestinal capsule. We have found that when a human subject, suffering from a gastric bloating sensation, ingests a vibrating gastrointestinal capsule according to a particular treatment schedule, and the capsule vibrates within the gastrointestinal tract of the subject in accordance with a vibration protocol, the gastric bloating sensation is reduced or alleviated.

We have further discovered a method for treating diarrhea in a human and reducing Bristol stool scores of fecal matter of the subject using an ingestible vibrating gastrointestinal capsule. We have found that when a human subject, suffering from chronic or periodic diarrhea and/or having fecal matter which is high on a Bristol stool scale, ingests a vibrating gastrointestinal capsule according to a particular treatment schedule, and the capsule vibrates within the gastrointestinal tract of the subject in accordance with a vibration protocol, the diarrhea is reduced or alleviated.

Methods of treating constipation using a vibrating gastrointestinal capsule are known in the art, and are described, for example, in U.S. Pat. No. 9,707,150. We have surprisingly found that similar vibrating gastrointestinal capsules are also useful in treating diarrhea, which, in some ways, is a condition opposite to that of constipation.

We have additionally discovered a method for treating gastroparesis in a human using an ingestible vibrating gastrointestinal capsule. We have found that when a human subject, suffering from gastroparesis, ingests a vibrating gastrointestinal capsule according to a particular treatment schedule, and the capsule vibrates within the gastrointestinal tract of the subject, and specifically within the stomach of the subject, in accordance with a vibration protocol, the gastroparesis is reduced or alleviated.

Methods of treating constipation using a vibrating gastrointestinal capsule are known in the art, and are described, for example, in U.S. Pat. No. 9,707,150. We have surprisingly found that similar vibrating gastrointestinal capsules are also useful in treating gastroparesis, which is a condition of the stomach, unlike constipation which is typically the result of an intestinal condition.

We have also discovered a method for reducing a straining sensation of a human subject during defecating and increasing Bristol stool scores of fecal matter of the subject using an ingestible vibrating gastrointestinal capsule. We have found that when a human subject, suffering from a straining sensation during defecating and/or having fecal matter which is low on a Bristol stool scale, ingests a vibrating gastrointestinal capsule according to a particular treatment schedule, and the capsule vibrates within the gastrointestinal tract of the subject in accordance with a vibration protocol, the straining sensation is reduced or alleviated and the Bristol stool score improves.

For the purposes of this application, the term “subject” relates to a human.

For the purposes of this application, the term “vibrating ingestible capsule” relates to an ingestible capsule adapted to at least intermittently vibrate, for a cumulative duration of at least one minute, in accordance with a vibration protocol of the capsule.

For the purposes of this application, the term “vibrating agitator” refers to any type of mechanism that vibrates or causes elements in its vicinity to vibrate, including a motor driven agitator such as a motor driven eccentric weight or a motor driven pendulum.

For the purposes of this application, the terms “intermittently activated vibrating agitator” and “intermittently activated vibrating agitation mechanism” refers to a vibration engine that vibrates and is operative at certain times, and does not vibrate at other times, the activation times being selected by a control element or other control unit controlling the vibration engine.

For the purposes of this application, the term “control element”, and the equivalent term “controller” refer to a component for controlling operation of mechanical and/or electrical components of the capsule, which includes a processing unit functionally associated with a non-tangible computer readable storage medium. The storage medium stores instructions, which, when executed by the processing unit, cause the processing unit to carry out actions which control the operation of the mechanical and/or electrical components of the capsule. For example, the instructions may include instructions to activate operation of a vibrating agitator at a specific time, frequency, cycle, and/or for a specific duration. The control element may be functionally associated with, or may include, a transceiver for receiving input, which input may be used to trigger execution of specific instructions stored in the storage medium.

For the purposes of this application, the term “vibration protocol” relates to a protocol specifying vibration parameters of an intermittently activated vibrating agitator of a vibrating ingestible capsule. Typically, the vibration protocol relates to an activation delay for initiating vibration (a duration between activation of the capsule and the first activation of the vibration engine), a vibration rate (number of vibration cycles per hour), a vibration duration and a repose duration for each vibration cycle, a vibration frequency, an amount of force exerted by the vibrations, and the like.

For the purposes of this application, the term “treatment procedure” relates to parameters of a treatment utilizing vibrating ingestible capsules, which are typically defined by a treating physician or medical practitioner. For example, the treatment procedure may include the number of capsules to be taken within a specific time duration (e.g. 3 capsules per week, 2 capsules per day), the frequency at which capsules should be taken, the time of day at which capsules should be taken, whether the capsule should be taken with or without food, and the like.

For the purpose of this application, the term “treatment protocol” relates to all aspects of treatment of a subject with a vibrating ingestible capsule, and includes the treatment procedure as well as the vibration protocol to be used for treating the subject.

For the purpose of the application, the term “transit time” relates to the amount of time it takes for a quanta of food or chyme to move a predetermined distance along the gastrointestinal tract of a particular subject. For example, the transit time may be the amount of time it takes a quanta of chyme to move from the duodenum to the rectum of the subject. The term transit time may relate to a transit time along the whole gastrointestinal tract, from the subject ingesting a food till chyme of that food reaches the rectum, or may relate to transit time within a segment of the gastrointestinal tract, such as the time it takes food from swallowing thereof till it passes from the stomach into the duodenum.

For the purpose of this application, the terms “treat diarrhea” and “reduce diarrhea” relate to providing a treatment, such that by the end of a treatment period, may be at least 2 weeks, at least 3 weeks, at least 4 weeks, at least 6 weeks, or at least 8 weeks, the frequency of diarrhea or loose bowel movements is reduced by at least 5%, at least 10%, at least 15%, at least 20%, or at least 25%. In some cases, the frequency of diarrhea is or loose bowel movements is reduced by at least one loose bowel movement per two weeks, at least one loose bowel movement per week, at least three loose bowel movements per two weeks, or at least two loose bowel movements per week.

For the purpose of this application, the term “alleviate diarrhea” relates to providing a treatment such that diarrhea ceases to be a chronic or persistent condition occurring on a daily or weekly basis.

For the purpose of this application, the terms “reduce Bristol stool score” relate to providing a treatment, such that by the end of a treatment period, may be at least 2 weeks, at least 3 weeks, at least 4 weeks, at least 6 weeks, or at least 8 weeks, a Bristol stool score of fecal matter, as sensed by the subject, is at least 1 point lower than at the beginning of the treatment period, in at least 5%, at least 10%, at least 15%, at least 20%, or at least 25% of the bowel movements.

For the purpose of this application, the terms “treat gastroparesis” and “reduce gastroparesis” relate to providing a treatment, such that by the end of the treatment period, which may be at least 2 weeks, at least 3 weeks, at least 4 weeks, at least 6 weeks, or at least 8 weeks, there is an improvement of at least 5%, at least 10%, at least 15%, or at least 20% in at least one symptom of gastroparesis over the entire treatment period. For example, a subject being nauseous at least 5% less of the time, a subject having at least 5% fewer vomiting episodes per week, a marker showing that at least 5% more food passed from the stomach into the intestine of the subject than had passed before initiation of treatment, and/or a marker showing at least 5% more contraction of the pyloric sphincter or in the vicinity of the pyloric sphincter than were measured prior to initiation of treatment, a subject having heart-burn or acid reflux at least 5% less of the time, or a subject able to consume at least 5% more food per day, are all considered successful treatment of gastroparesis or reduction of gastroparesis.

For the purpose of this application, the term “alleviate gastroparesis” relates to providing a treatment such that gastroparesis ceases to be a chronic or persistent condition occurring on a daily or weekly basis, such that food is pumped from the stomach into the intestine on a regular basis.

For the purpose of this application, the terms “alleviate sensation of straining” and “dissipate sensation of straining” relate to providing a treatment, such that by the end of a treatment period, may be at least 2 weeks, at least 3 weeks, at least 4 weeks, at least 6 weeks, or at least 8 weeks, the sensation of straining sensed by the subject is at least one point lower than at the beginning of the treatment period, and/or a sensation of straining while defecating occurs 5% to 15% fewer times than at the beginning of the treatment period.

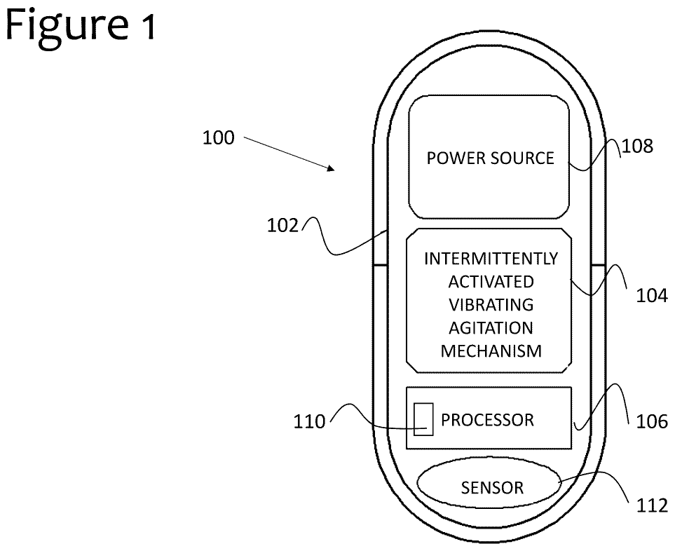

For the purpose of this application, the terms “increase Bristol stool score” relate to providing a treatment, such that by the end of a treatment period, may be at least 2 weeks, at least 3 weeks, at least 4 weeks, at least 6 weeks, or at least 8 weeks, a Bristol stool score of fecal matter, as sensed by the subject, is at least 1 point higher than at the beginning of the treatment period, in at least 5%, at least 10%, at least 15%, at least 20%, or at least 25% of the bowel movements. Referring now to the drawings, FIG. 1 is a schematic block diagram of a vibrating ingestible capsule for reducing a straining sensation of a human subject during defecating and improving Bristol stool scores of fecal matter of the subject according to embodiments of the present invention.

Referring now to the drawings, FIG. 1 is a schematic block diagram of an ingestible vibrating gastrointestinal capsule for treating a gastric bloating sensation within a stomach or midriff region of a human subject according to embodiments of the present invention.

As seen in FIG. 1, vibrating ingestible capsule 100 includes a capsule housing or shell 102, having disposed therein a vibrating agitator 104 adapted to be intermittently activated, a processor, or control element 106 adapted to control operation of the vibrating agitator 104, and at least one power source 108 providing power to vibrating agitator 104 and processor 106. In some embodiments, the processor is functionally associated with a timer 110. In some embodiments, capsule 100 further includes at least one sensor 112, functionally associated with processor 106. The at least one sensor 112 may be adapted to sense at least one parameter within capsule 100 or in an environment of capsule 100, and may include a temperature sensor, a moisture sensor, a pH sensor, or any other suitable sensor.

Power source 108 may be any suitable power source, such as one or more alkaline or silver oxide batteries, primary batteries, rechargeable batteries, capacitors and/or supercapacitors. In some embodiments, the power source may be a zinc-manganese dioxide alkaline battery, such as a AG3/LR41 button cell, commercially available from Daly-Station Battery Limited of Shenzhen Guandong, P.R. China.

Intermittently activated vibrating agitator 104 is adapted to have a vibration mode of operation and a rest mode of operation. In the vibration mode of operation, intermittently activated vibrating agitator 104 is adapted to exert forces on capsule housing 102, such that capsule housing 102 exerts vibrations on an environment surrounding capsule 100.

In some embodiments, intermittently activated vibrating agitator 104 may include a radial agitation mechanism adapted to exert radial forces on capsule housing 102, in a radial direction with respect to a longitudinal axis of housing 102. For example, the radial agitation mechanism may include an unbalanced weight attached to a shaft of an electric motor powered by said battery, substantially as described in U.S. Pat. No. 9,707,150, which is incorporated by reference for all purposes as if fully set forth herein.

In some embodiments, intermittently activated vibrating agitator 104 may include an axial agitation mechanism adapted to exert axial forces on the capsule housing 102, in an axial direction with respect to a longitudinal axis of housing 102. For example, the axial agitation mechanism may include an electric motor powered by the battery and an urging mechanism, associated with, and driven by, the electric motor, such that the urging mechanism adapted to exert said axial forces, substantially as described in U.S. Pat. No. 9,707,150. In some embodiments, the urging mechanism adapted to exert the axial forces in opposite directions. In some embodiments, the urging mechanism is adapted to deliver at least a portion of the axial forces in a knocking mode.

In some embodiments, the forces exerted by intermittently activated vibrating agitator 104 on capsule housing 102 in the vibration mode of operation include radial forces in a radial direction with respect to the longitudinal axis of the housing and axial forces in an axial direction with respect to the longitudinal axis. In some embodiments, a single agitation mechanism exerts both the radial and the axial forces. In other embodiments, the axial forces are exerted by one agitation mechanism, and the radial forces are exerted by another, separate, agitation mechanism, where both agitation mechanisms form part of intermittently activated vibrating agitator 104.

In some embodiments, intermittently activated vibrating agitator 104 may be a coin type eccentric vibration motor, such as a coin-type motor having the Product Part No. C0834L-066332017-2001 commercially available from Ineed HK Limited of Kowloon, Hong-Kong.

In the vibrating mode of operation, intermittently activated vibrating agitator 104 is adapted to have a plurality of vibration cycles, where each cycle includes a vibration duration followed by a repose duration. Forces are exerted by the vibrating agitator 104 on capsule housing 102 only during the vibration duration, and as such capsule housing 102 only exerts forces on an environment thereof during the vibration duration.

In some embodiments, the number of vibration cycles per hour is in the range of 20 to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, 5 seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 108.

It will be appreciated by persons skilled in the art that the vibration mode of operation may be intermittent, or interrupted, such that vibrating agitator 104 is operative in the vibration mode for a first duration, for example 30 minutes, then does have any vibration cycles for a second duration, for example 1 hour, and then is operative in the vibration mode and has vibration cycles for a third duration, for example two hours. The cumulative duration relates to the sum of all durations during which vibrating agitator 104 was operative in the vibration mode and included vibration cycles, including the vibration duration and the repose duration of the vibration cycle.

In some embodiments, vibrating agitator 104 is configured to exert forces on the capsule housing 102, such that a net force exerted by the capsule housing 102 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitator 104 is configured to exert said forces on capsule housing 102 to attain a capsule housing 102 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source and of the vibrating agitator.

It will be further appreciated that a specific capsule may be controlled by the processor such that different vibrational frequencies may be attained and/or different net forces may be exerted, by the capsule, in different vibration cycles of the capsule. Due to the natural distinction between subjects, use of multiple different parameters in different vibration cycles of a single capsule would allow the capsule to successfully treat multiple subjects, even if the personal optimal treatment for those subjects is not the same, as there is a higher chance that in at least some of the vibration cycles the activation parameters of the capsule would reach, or be close to, the optimal parameters for each specific subject.

Processor 106 is adapted to control the operation of intermittently activated vibrating agitator 104. Such control may include control of any one or more of the force applied by the vibrating agitator, the vibrational frequency reached, the times in which vibrating agitator 104 operates in the vibration mode of operation, the vibration duration of each vibration cycle, the repose duration of each vibration cycle, the vibration cycle duration, and cumulative vibration duration of the vibrating agitators.

In some embodiments, processor 106 is adapted to wait for a pre-set activation time delay following activation of capsule 100 and prior to initiation of the vibration mode of operation of vibrating agitator 104. The activation time delay may be any suitable time delay, and may be dependent on portions of the gastrointestinal tract in which it is desired that the capsule will operate.

For example, in embodiments in which it is desired that the capsule operate, or vibrate, in an intestinal portion, such as for treatment of straining, the activation time delay may be in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

As another example, in embodiments in which it is desired that the capsule operate, or vibrate, within the stomach of the subject, such as for treatment of gastroparesis, the activation time delay may be in the range of 1 minute to 6 hours, 1 minute to 5 hours, 1 minute to 4 hours, 1 minute to 3 hours, 1 minute to 2 hours, 5 minutes to 6 hours, 5 minutes to 5 hours, 5 minutes to 4 hours, 5 minutes to 3 hours, 5 minutes to 2 hours, 10 minutes to 6 hours, 10 minutes to 5 hours, 10 minutes to 4 hours, 10 minutes to 3 hours, or 10 minutes to 2 hours

In some embodiments, processor 106 is adapted to receive information relating to the desired vibration protocol from a control unit (not shown), prior to ingestion of the capsule or to activation thereof. For example, the information may be remotely transmitted from the control unit to processor 106, for example using a short range wireless communication method. In some embodiments, the information is transmitted as a list of vibration parameters for effecting the vibration protocol. In some embodiments, the information is transmitted as executable code for effecting the vibration protocol.

In some embodiments, the information includes one or more of a desired activation time delay, a desired number of vibration cycles, a desired vibration duration in each vibration cycle, a desired repose duration in each vibration cycle, a desired cumulative vibration duration, and the like.

In some embodiments, processor 106, or a timer associated therewith, is adapted to be activated by the control unit prior to ingestion of capsule 100. In some embodiments, activation is carried out by sending a signal to processor 106, for example using a short range wireless communication protocol. In some embodiments, the activation signal activates the timer to immediately begin effecting the vibration protocol. In some embodiments, the at least one sensor 108 is adapted to identify ingestion of the capsule, and processor 106 is adapted to begin effecting the vibration protocol immediately following identification of ingestion of capsule 100.

In some embodiments, processor 106 is adapted to control vibrating agitator 104 so that the capsule applies forces to an environment thereof to effect a mechanical stimulation of the wall of the gastrointestinal tract of the subject in a targeted zone.

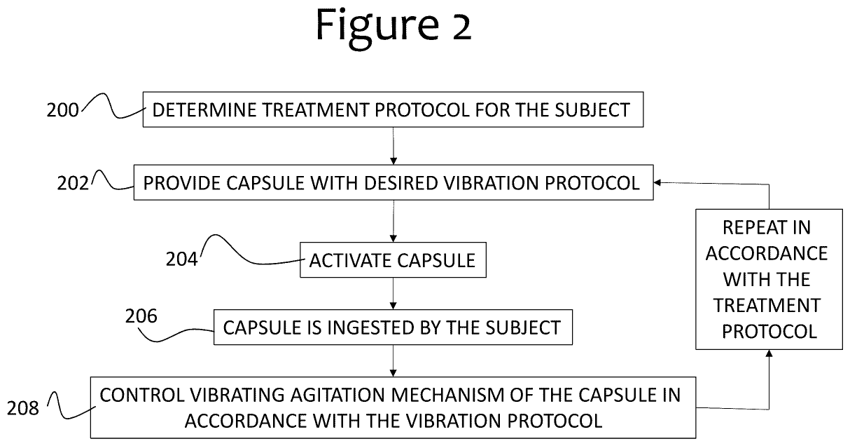

Reference is now additionally made to FIG. 2, which is a schematic flowchart of a method for treating a condition of the gastrointestinal tract of a human subject according to the present invention, the treatment being based one use of an ingestible vibrating gastrointestinal capsule such as capsule 100 of FIG. 1. In some embodiments, the condition may be, or may include, a gastric bloating sensation within a stomach or midriff region of the human subject. In some embodiments, the condition may be, or may include, diarrhea. In some embodiments the condition may be, or may include, gastroparesis.

As seen at step 200, initially the treatment protocol for the subject is set or determined, for example, by a treating physician or medical practitioner. The treatment protocol may indicate the number of treatment sessions per week or per other time duration, the time of day at which a capsule should be ingested, a targeted zone in which the capsule should be operative, and/or may indicate the vibration protocol of the capsule. For treatment of gastroparesis, the targeted zone may within the stomach of the subject, or in an area in the vicinity of the pyloric sphincter.

At step 202, the processor 106 of an ingestible capsule 100 may optionally receive, or be programmed with, a desired vibration protocol in accordance with the treatment protocol determined at step 200. In some embodiments, such programming of the desired vibration protocol is effected by a control unit. For example, the programming may include remotely transmitting the desired vibration protocol from the control unit to the processor 106, for example using a short range wireless communication method. In some embodiments, the desired vibration protocol is transmitted as a list of vibration parameters for effecting the vibration protocol. In some embodiments, the desired vibration protocol is transmitted as executable code for effecting the vibration protocol.

In some embodiments, step 202 includes pre-setting of an activation time delay for activation of the capsule. However, in some embodiments, ingestible capsule 100 may be pre-programmed, for example with a default vibration protocol or with a pre-set protocol, in which case, step 202 may have been previously executed, e.g., by the capsule manufacturer.

The vibration protocol pre-set or programmed into the capsule 100, and specifically the activation time delay of the capsule, is selected to effect vibration of the capsule 100 when the capsule will be located in a targeted zone within the gastrointestinal tract of the subject. In some embodiments, the targeted zone is defined in the treatment protocol determined at step 200.

In some embodiments, the targeted zone is an intestinal section of the gastrointestinal tract of the subject, such as a section of the small intestine, the large intestine, and/or the rectum. In other embodiments, such as embodiments that include treatment of gastroparesis, the targeted zone is within the stomach of the subject or adjacent the pyloric sphincter.

In embodiments in which the targeted zone includes an intestinal section of gastrointestinal tract, the activation time delay is selected to be in the range of 2 hours to 48 hours, 2 hours to 42 hours, 2 hours to 36 hours, 2 hours to 30 hours, 2 hours to 24 hours, 3 hours to 24 hours, 4 hours to 24 hours, 4 hours to 20 hours, 4 hours to 18 hours, 4 hours to 16 hours, 4 hours to 14 hours, 4 hours to 12 hours, 6 hours to 12 hours, or 6 hours to 10 hours.

In embodiments in which the targeted zone is in the stomach of the subject, the activation time delay is selected to be in the range of 1 minute to 6 hours, 1 minute to 5 hours, 1 minute to 4 hours, 1 minute to 3 hours, 1 minute to 2 hours, 5 minutes to 6 hours, 5 minutes to 5 hours, 5 minutes to 4 hours, 5 minutes to 3 hours, 5 minutes to 2 hours, 10 minutes to 6 hours, 10 minutes to 5 hours, 10 minutes to 4 hours, 10 minutes to 3 hours, or 10 minutes to 2 hours.

In some embodiments, the selected activation time delay is selected according to a transit time of chyme along the gastrointestinal tract of the subject being treated. In some such embodiments, information relating to the transit time of chyme is collected prior to step 202.

The capsule is activated for use at step 204. In some embodiments, activation is performed automatically when the capsule receives the vibration protocol, at step 202. In other embodiments, such as in embodiments in which the vibration protocol is pre-set, the capsule may be explicitly activated, such as by receipt of an activation signal from the control unit or by sensors within the capsule sensing that the capsule has been ingested. Activation of the capsule results in activation of the timer associated with the processor 106, and is the start of the activation time delay.

Following activation of capsule 100, or together therewith, capsule 100 is ingested by the subject, and begins to travel through the gastrointestinal tract of the subject, as seen at step 206.

At step 208, while capsule 100 is travelling in the gastrointestinal tract together with the food/chyme therein, processor 106 controls the vibrating agitator 104 in accordance with the vibration protocol, so that vibrating agitator 104 is in the vibrating mode of operation when the capsule is disposed in the targeted zone.

Operation of vibrating agitator 104 in the vibrating mode of operation effects vibration of capsule housing 102, as described hereinabove, such that the housing exerts vibrations on the environment surrounding the capsule in the targeted zone. Specifically, vibration of capsule housing 102 may be intended to effect a mechanical stimulation of the wall of the gastrointestinal tract in the targeted zone.

A treatment session as defined in steps 202 to 208 may be repeatedly administered to the subject as specified in the treatment protocol for the subject, determined or obtained at step 200. In some embodiments, the treatment protocol includes administering a plurality of treatment sessions to the subject. In some embodiments, the treatment protocol includes administering at least one treatment session to the subject per week, over a treatment period of at least two weeks, at least at least three weeks, at least four weeks, at least five weeks, at least six weeks, or at least eight weeks. In some embodiments, the treatment protocol includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

In some embodiments, the condition of the gastrointestinal tract being treated is, or includes, a sensation of gastric bloating. In such embodiments, the subject may be any suitable subject, suffering from a sensation of gastric bloating. The sensation of gastric bloating may be caused by any of a number of underlying conditions, such as chronic constipation, food allergies or intolerances, and/or hormonal or enzymatic deficiencies.

In some embodiments, the subject suffering from a sensation of gastric bloating is a subject who has experienced at most one of the following symptoms over the 3 months preceding the beginning of treatment:
fewer than three bowel movements per week;
straining;
lumpy or hard stools;
sensation of anorectal obstruction;
sensation of incomplete defecation; and
manual maneuvering required to defecate.

In some embodiments, the subject suffering from a sensation of gastric bloating is a subject who has experienced at most one of the following symptoms over the 3 months preceding the beginning of treatment:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

In some embodiments, the subject suffering from a sensation of gastric bloating is a constipation free subject.

In some embodiments, the subject suffering from a sensation of gastric bloating suffers from at least one of the following symptoms:
irritable bowel syndrome;
at least one food allergy;
at least one food intolerance;
enzymatic deficiency;
hormonal deficiency; and
hormonal imbalance.

In some embodiments, the condition of the gastrointestinal tract being treated is, or includes, diarrhea. In such embodiments, the subject may be any suitable subject suffering from chronic, persistent, or periodic diarrhea, and/or whose fecal matter has Bristol stool scores of 5 or more. The diarrhea may be caused by any of a number of underlying conditions, such as irritable bowel syndrome, inflammatory bowel disease such as Crohn's disease or ulcerative colitis, intestinal infections, hyperthyroidism, food allergies or intolerances, substance abuse, diabetes, and/or medications.

In some embodiments, the subject suffering from diarrhea is a subject which has at least two loose bowel movements per day, for at least two weeks prior to the beginning of treatment, or at least one loose bowel movement per day for at least one week prior to the beginning of treatment, where loose bowel movements are bowel movements having a Bristol stool score of 5 or more.

In some embodiments, the condition of the gastrointestinal tract being treated is, or includes, gastroparesis. In such embodiments, the subject may be any suitable subject suffering from gastroparesis. The gastroparesis may be caused by any of a number of underlying conditions, such as diabetes, injury to the vagus nerve, Parkinson's disease, multiple sclerosis, amyloidosis, scleroderma, substance abuse, and/or medications taken by the subject.

In some embodiments, the subject suffering from gastroparesis is a subject who has experienced nausea at least 25% of the time for at least two weeks preceding treatment.

In some embodiments, the subject suffering from gastroparesis is a subject who has experienced vomiting after at least one meal per day or after 25% of the meals for at least two weeks preceding treatment.

In some embodiments, the subject suffering from gastroparesis is a subject who has experienced a feeling of fullness after eating ten bites or fewer in at least 50% of the meals for at least two weeks preceding treatment.

In some embodiments, the subject suffering from gastroparesis is a subject who has experienced heart burn or acid reflux at least 25% of the time for at least two weeks preceding treatment.

In some embodiments, the condition of the gastrointestinal tract being treated is, or includes, a sensation of straining during defecating. In such embodiments, the subject may be any suitable subject, suffering from a sensation of straining during defecating and/or whose fecal matter has a low score on the Bristol stool scale. The sensation of straining during defecating and/or a low score on the Bristol stool scale may be caused by any of a number of underlying conditions, such as chronic constipation, food allergies or intolerances, hormonal or enzymatic deficiencies, irritable bowel syndrome, colon cancer, medications taken by the subject which change the consistency of the fecal matter, neurological diseases, and hypothyroidism.

In some embodiments, the subject suffering from a sensation of straining during defecating is a subject who has experienced at most one of the following symptoms over the 3 months preceding the beginning of treatment:
fewer than three bowel movements per week;
straining;
lumpy or hard stools;
sensation of anorectal obstruction;
sensation of incomplete defecation; and
manual maneuvering required to defecate.

In some embodiments, the subject suffering from a sensation of straining during defecating is a subject who has experienced at most two of the following symptoms over the 3 months preceding the beginning of treatment:
fewer than three bowel movements per week;
straining;
lumpy or hard stools;
sensation of anorectal obstruction;
sensation of incomplete defecation; and
manual maneuvering required to defecate.

In some embodiments, the subject suffering from a sensation of straining during defecating is a subject who has experienced at most one of the following symptoms over the 3 months preceding the beginning of treatment:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

In some embodiments, the subject suffering from a sensation of straining during defecating is a constipation free subject.

In some embodiments, the subject suffering from a sensation of straining during defecating suffers from emotional stress and/or from psychosomatically caused symptoms.

### EXAMPLES

Reference is now made to the following examples, which, together with the above description, illustrates the invention in a non-limiting fashion.

### Example 1

A study was conducted in which 24 participating subjects suffering from a gastric bloating sensation were treated with a vibrating gastrointestinal capsule according to a treatment protocol, in accordance with the present invention.

The treatment protocol included treatment cycles including administering one vibrating gastrointestinal capsule per day for two days, followed by one day where no capsule is administered, repeated for a treatment duration of six weeks. At the end of each week of treatment, as well as at the end of a two week run-in period preceding the initiation of treatment, the subjects were asked to rank the degree to which they felt a sensation of gastric bloating, on a scale of 1 to 10, where 10 represents a severe gastric bloating sensation and 1 represents a very mild or infrequent gastric bloating sensation.

The administered capsules included a zinc-manganese dioxide alkaline battery, such as a AG3/LR41 button cell, commercially available from Daly-Station Battery Limited of Shenzhen Guandong, P.R. China, as the power source, and a coin-type eccentric vibration motor, such as a coin-type motor having the Product Part No. C0834L-066332017-2001, commercially available from Ineed HK Limited of Kowloon, Hong-Kong, as the vibrating agitator.

The administered capsules were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. During the vibration mode of operation, the force applied by the capsule housing on the surrounding environment was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

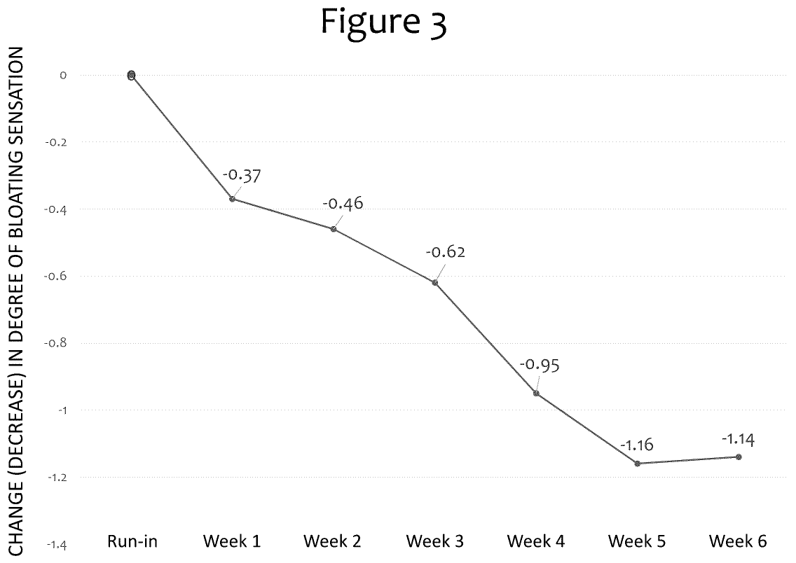

The results of the study are shown in FIG. 3, which illustrates the improvement in the average degree of gastric bloating sensed by the participating subject vs. treatment time. As seen in FIG. 3, the end of the run-in period sets the baseline measure for the average degree of gastric bloating sensed by participants (and as such shows 0 improvement relative to baseline). During the treatment period, the average degree of gastric bloating sensed by participants appears to monotonically improve, until at the end of the 6-week treatment period, the average degree of gastric bloating sensed by the participants was more than one ranking lower, indicating a significant improvement in the degree of gastric bloating sensed by the participants. As such, the results illustrated in FIG. 3 are indicative of the success of the treatment of the present invention.

### Example 2

A study which included 150 participating subjects suffering from a gastric bloating sensation was conducted. Half of the participating subjects, termed herein “trial subjects”, were treated with a vibrating gastrointestinal capsule according to a treatment protocol, in accordance with the present invention, while the other half, termed herein “sham subjects”, were treated with a sham capsule, which appeared and behaved identically to the vibrating gastrointestinal capsule prior to ingesting thereof, but did not vibrate within the subject's alimentary tract.

The treatment protocol included treatment cycles including administering one capsule per day for two days, followed by one day where no capsule is administered, repeated for a treatment duration of six weeks, where the trial subjects received a vibrating gastrointestinal capsule, and the sham subjects received a sham capsule.

At the end of each week of treatment, as well as at the end of a two week run-in period preceding the initiation of treatment, the subjects were asked to rank the degree to which they felt a sensation of gastric bloating, on a scale of 1 to 10, where 10 represents a severe gastric bloating sensation and 1 represents a very mild or infrequent gastric bloating sensation.

The administered capsules included a zinc-manganese dioxide alkaline battery, such as a AG3/LR41 button cell, commercially available from Daly-Station Battery Limited of Shenzhen Guandong, P.R. China, as the power source, and a coin-type eccentric vibration motor, such as a coin-type motor having the Product Part No. C0834L-066332017-2001, commercially available from Ineed HK Limited of Kowloon, Hong-Kong, as the vibrating agitator.

The capsules administered to the trial subjects were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. During the vibration mode of operation, the force applied by the capsule housing on the surrounding environment was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

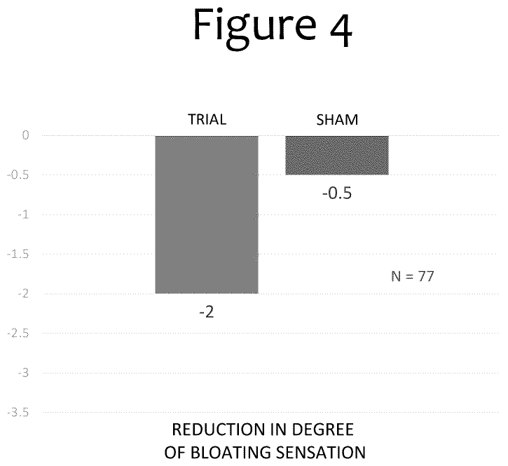

FIG. 4 illustrates the improvement in the average degree of gastric bloating sensed by the participating subjects at the end of the study for 50% of the participating subjects having moderate to severe gastric bloating, and removing subjects suffering from mild gastric bloating and from very severe gastric bloating. Stated differently, the results illustrated in FIG. 4 relate to the 50% of the participants at the center of the Gaussian curve of gastric bloating severity, for both the trial subjects and the sham subjects.

As seen in FIG. 4, at the end of the 6-week treatment period, on average, the average degree of gastric bloating sensed by the trial subjects having moderate to severe gastric bloating was two full rankings lower, indicating a significant improvement in the degree of gastric bloating sensed by those subjects. By contrast, the average degree of gastric bloating sensed by the sham subjects was half a ranking lower. As such, the results illustrated in FIG. 4 indicate that for a statistically-significant subject pool, (i) the inventive treatment method appreciably alleviated gastric bloating sensations, and (ii) the favorable results are well above and beyond the more minor degree of alleviation associated with placebo effects.

### Example 3

A study was conducted, in which 24 participating subject suffering from a sensation of straining while defecating and whose fecal matter had a Bristol stool score of 1-2 were treated with a vibrating gastrointestinal capsule according to a treatment protocol.

The treatment protocol included treatment cycles including administering one vibrating gastrointestinal capsule per day for two days, followed by one day where no capsule is administered, repeated for a treatment duration of six weeks.

At the end of each week of treatment, as well as at the end of a two week run-in period preceding the initiation of treatment, the subjects were asked to rank the degree to which they felt a sensation of straining during defecating, on a scale of 1 to 11, where 11 represents a severe straining sensation and 1 represents a very mild or infrequent straining sensation. Additionally, at the beginning of the run-in period, at the beginning of the treatment, at the midpoint of the treatment, and at the end of the treatment, the subjects were asked to indicate the Bristol stool score of fecal matter thereof, based on a Bristol stool chart.

The administered capsules included a zinc-manganese dioxide alkaline battery, such as a AG3/LR41 button cell, commercially available from Daly-Station Battery Limited of Shenzhen Guandong, P.R. China, as the power source, and a coin type eccentric vibration motor, such as a coin-type motor having the Product Part No. C0834L-066332017-2001 commercially available from Ineed HK Limited of Kowloon, Hong-Kong, as the vibrating agitator.

The administered capsules were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. The force applied by the capsule housing to the environment therearound during the vibration mode of operation was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

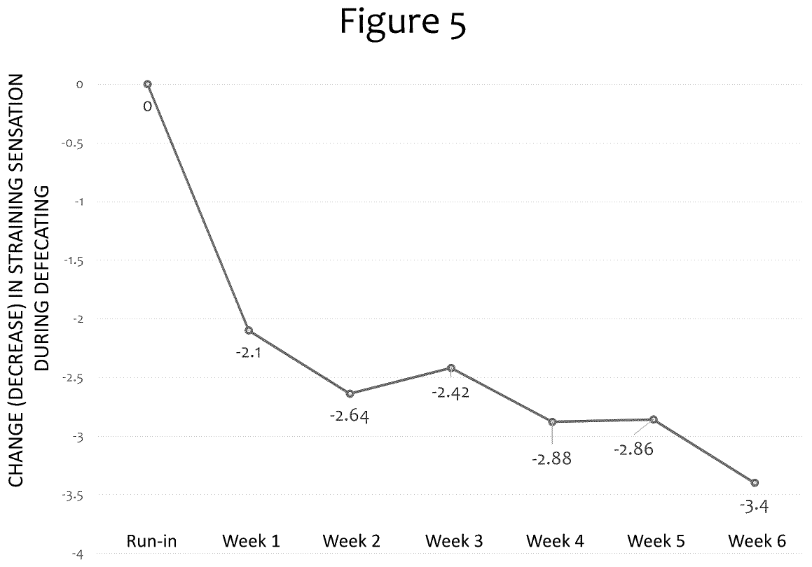

The results of the study with respect to the sensation of straining during defecating are shown in FIG. 5, which illustrates the improvement in the average degree of straining sensed by the participating subjects vs. treatment time. As seen in FIG. 5, the end of the run-in period sets the baseline measure for the average degree of straining sensed by participants, and as such shows 0 improvement relative to baseline. During the treatment period, the average degree of straining sensed by participants appears to monotonically improve, until at the end of the 6-week treatment period, the average degree of straining during defecating sensed by the participants was more than three rankings lower, indicating a significant improvement in the degree of straining during defecating sensed by the participants. As such, the results illustrated in FIG. 5 are indicative of the success of the treatment of the present invention in reducing the sensation of straining during defecating.

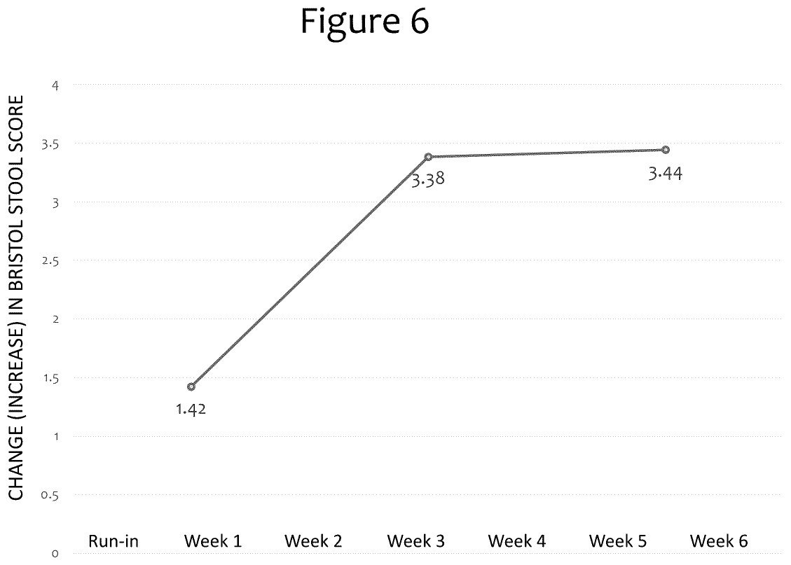

The results of the study with respect to a Bristol stool score of fecal matter are shown in FIG. 6, which illustrates the average Bristol stool score indicated by the participating subjects vs. treatment time. As seen in FIG. 6, at the beginning of treatment, the average Bristol stool score was less than 1.5, indicative of hard stool which is difficult to pass. An improvement was felt by the participants by the trial midpoint, after three weeks of treatment, when the average Bristol stool score was 3.38, indicative of normal stool. The average Bristol stool score remained within the normal range (between 3 and 4) until the end of the treatment period, after 6 weeks. As such, the results illustrated in FIG. 6 are indicative of the success of the treatment of the present invention in increasing the Bristol stool score of fecal matter by 2 points on a 7 points scale, for subjects treated by the method of FIG. 2, and specifically, that fecal matter of such subjects, on average, moved from being hard to pass to being normal.

### Example 4

A study which included 150 participating subjects suffering from a sensation of straining while defecating and whose fecal matter had a Bristol stool score of 1-2 was conducted. Half of the participating subjects, termed herein “trial subjects”, were treated with a vibrating gastrointestinal capsule according to a treatment protocol, in accordance with the present invention, while the other half, termed herein “sham subjects”, were treated with a sham capsule, which appeared and behaved identically to the vibrating gastrointestinal capsule prior to ingesting thereof, but did not vibrate within the subject's alimentary tract.

The treatment protocol included treatment cycles including administering one capsule per day for two days, followed by one day where no capsule is administered, repeated for a treatment duration of six weeks, where the trial subjects received a vibrating gastrointestinal capsule, and the sham subjects received a sham capsule.

At the end of each week of treatment, as well as at the end of a two week run-in period preceding the initiation of treatment, the subjects were asked to rank the degree to which they felt a sensation of straining during defecating, on a scale of 1 to 11, where 11 represents a severe straining sensation and 1 represents a very mild or infrequent straining sensation. Additionally, at the beginning of the run-in period, at the beginning of the treatment, at the midpoint of the treatment, and at the end of the treatment, the subjects were asked to indicate the Bristol stool score of fecal matter thereof, based on a Bristol stool chart.

The administered capsules included a zinc-manganese dioxide alkaline battery, such as a AG3/LR41 button cell, commercially available from Daly-Station Battery Limited of Shenzhen Guandong, P.R. China, as the power source, and a coin-type eccentric vibration motor, such as a coin-type motor having the Product Part No. C0834L-066332017-2001, commercially available from Ineed HK Limited of Kowloon, Hong-Kong, as the vibrating agitator.

The capsules administered to the trial subjects were programmed to have a activation time delay of 8 hours, and, when in the vibration mode of operation, to have vibration treatment cycles including a 3 second vibration duration followed by a 16 second repose duration, for a cumulative treatment duration of 2.5 to 3 hours. During the vibration mode of operation, the force applied by the capsule housing on the surrounding environment was in the range of 200 gram-force to 500 gram-force, and the vibrational frequency was in the range of 120 Hz to 250 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

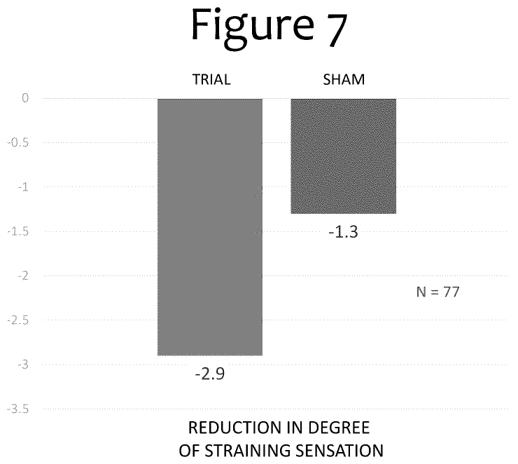

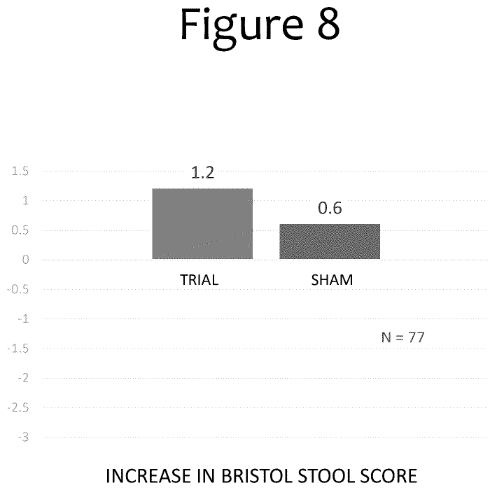

FIGS. 7 and 8 illustrates the improvement in the average degree of straining sensed by the participating subjects at the end of the study and the improvement in the average Bristol stool score for 50% of the participating subjects having moderate to severe symptoms, and removing subjects suffering from mild symptoms and from very severe symptoms. Stated differently, the results illustrated in FIGS. 7 and 8 relate to the 50% of the participants at the center of the Gaussian curve of symptom severity for both the trial subjects and the sham subjects.

As seen in FIG. 7, at the end of the 6-week treatment period, the average degree of straining during defecating sensed by the trial subjects having moderate to severe symptoms was almost three whole rankings (2.9 rankings) lower, indicating a significant improvement in the degree of straining during defecating sensed by those subjects. By contrast, the average degree of straining during defecating sensed by the sham subjects was 1.3 ranking lower. As such, the results illustrated in FIG. 7 indicate that for a statistically-significant subject pool, (i) the inventive treatment method appreciably alleviated straining sensations during defecating, and (ii) the favorable results are well above and beyond the more minor degree of alleviation associated with placebo effects.

As seen in FIG. 8, at the end of the 6-week treatment period, the average Bristol stool score of fecal matter as sensed by the trial subjects having moderate to severe symptoms was 1.2 rankings higher, indicating a significant improvement in the Bristol stool score of fecal matter of those subjects, who, at the start of the trial, had very low Bristol stool scores. By contrast, the average Bristol stool score of fecal matter as sensed by the sham subjects was, on average, 0.6 rankings higher. As such, the results illustrated in FIG. 8 indicate that for a statistically-significant subject pool, (i) the inventive treatment method appreciably increases the Bristol stool score of subjects initially having a low Bristol stool score, and (ii) the favorable results are well above and beyond the more minor degree of improvement associated with placebo effects.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of treating two opposite sensations within a stomach or midriff region of a human subject, or two opposite conditions of the gastrointestinal tract of the human subject, the method comprising:
(a) providing, to the human subject suffering from one of the two opposite sensations or one of the two opposite conditions, a vibrating gastrointestinal capsule adapted to transit an alimentary canal of a human, said vibrating gastrointestinal capsule having:
a housing;
a battery, disposed within said housing; and
a vibrating agitator, powered by said battery, said vibrating agitator adapted such that, in a first vibrating mode of operation, said housing exerts vibrations on an environment surrounding said vibrating gastrointestinal capsule, said vibrating gastrointestinal capsule being controllable to effect said first vibrating mode of operation; and
(b) in response to the human subject feeling one of the two opposite sensations or suffering from one of the two opposite conditions, ingesting, by the human subject, said vibrating gastrointestinal capsule, to treat said one of the two opposite sensations or said one of the two opposite conditions,
wherein a frequency of vibration employed by said vibrating ingestible capsule during treatment of said two opposite conditions or said two opposite sensations, within the gastrointestinal tract of the human subject, is in the range of 100 Hz to 650 Hz, regardless of which of the two opposite conditions or the two opposite sensations is experienced by the human subject.

2. The method of claim 1, wherein a targeted zone at which vibration is applied by said vibrating ingestible capsule during treatment of said two opposite conditions or said two opposite sensations is the same regardless of which of the two opposite conditions or the two opposite sensations is experienced by the human subject.

3. The method of claim 1, wherein the two opposite conditions comprise diarrhea and constipation, and wherein said vibrating gastrointestinal capsule is adapted to treat diarrhea and constipation using said same treatment protocol.

4. The method of claim 1, wherein a first condition of said two opposite conditions is manifested by hard stool defecation and a second condition of said two opposite conditions is manifested by soft stool defecation, wherein said hard stool defecation is characterized by a stool having a first Bristol stool score lower than 2 and said soft stool defecation is characterized by a stool having a second Bristol stool score higher than 5, and wherein said vibrating gastrointestinal capsule is adapted to increase said first Bristol stool score and to decrease said second Bristol stool score using said same treatment protocol.

5. A method of treating a sensation of gastric bloating within a stomach or midriff region of a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator of said gastrointestinal capsule such that said first vibrating mode of operation occurs when said capsule is disposed within a targeted zone within a gastrointestinal tract of the subject, so as to alleviate or dissipate the sensation of gastric bloating.

6. The method of claim 5, wherein the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

7. The method of claim 5, wherein the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

8. The method of claim 5, wherein the subject is a constipation-free subject.

9. The method of claim 8, wherein the subject has at least one of:
irritable bowel syndrome;
at least one food allergy;
at least one food intolerance;
enzymatic deficiency;
hormonal deficiency; and
hormonal imbalance.

10. A method of treating at least one of diarrhea and gastroparesis in a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator such that said first vibrating mode of operation occurs when said capsule is disposed within a targeted zone within a gastrointestinal tract of the subject, so as to treat, reduce, or alleviate diarrhea in said subject.

11. A method of reducing a Bristol stool score of fecal matter defecated by a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator such that said first vibrating mode of operation occurs when said capsule is disposed within a targeted zone within a gastrointestinal tract of the subject, so as to reduce the Bristol stool score of the fecal matter defecated by the subject.

12. The method of claim 11, wherein prior to said method, said fecal matter has a Bristol stool score of at least 5.

13. The method of any one of claims 10 to 12, wherein the subject is a subject who has experienced at least three loose bowel movements daily for at least two weeks preceding treatment.

14. The method of any one of claims 10 to 12, wherein the subject is a subject who has experienced at least one loose bowel movement daily for at least one week preceding treatment.

15. The method of any one of claims 10 to 14, wherein said subject suffers from at least one of:
irritable bowel syndrome;
inflammatory bowel disease;
Crohn's disease;
ulcerative colitis;
intestinal infections;
hyperthyroidism;
at least one food allergy;
at least one food intolerance;
substance abuse; and
diabetes.

16. A method of treating gastroparesis in a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator such that said first vibrating mode of operation occurs when said capsule is disposed within a targeted zone within a gastrointestinal tract of the subject, so as to treat, reduce, or alleviate gastroparesis in said subject.

17. The method of claim 16, wherein the subject is a subject who has experienced nausea at least 25% of the time for at least two weeks preceding treatment.

18. The method of claim 16 or claim 17, wherein the subject is a subject who has experienced vomiting after at least one meal per day or after 25% of the meals for at least two weeks preceding treatment.

19. The method of any one of claims 16 to 18, wherein the subject is a subject who has experienced a feeling of fullness after eating ten bites or fewer in at least 50% of the meals for at least two weeks preceding treatment.

20. The method of any one of claims 16 to 19, wherein the subject is a subject who has experienced heart burn or acid reflux at least 25% of the time for at least two weeks preceding treatment.

21. The method of any one of claims 16 to 20, wherein said subject suffers from at least one of:
diabetes;
injury to the vagus nerve;
Parkinson's disease;
multiple sclerosis;
amyloidosis;
scleroderma;
substance abuse; and
side effects of medications taken by the subject.

22. A method of treating a sensation of straining while defecating in a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator such that said first vibrating mode of operation occurs when said capsule is disposed within a targeted zone within a gastrointestinal tract of the subject, so as to alleviate or dissipate the sensation of straining while defecating.

23. A method of increasing a Bristol stool score of fecal matter defecated by a human subject using a gastrointestinal capsule adapted to transit an alimentary canal of the subject, said capsule having:
a housing;
a battery, disposed within said housing; and
a vibrating agitator, powered by said battery, said vibrating agitator adapted such that, in a first vibrating mode of operation, said housing exerts vibrations on an environment surrounding said capsule, the method comprising:
(a) ingesting said gastrointestinal capsule; and
(b) controlling said vibrating agitator such that said first vibrating mode of operation occurs when said capsule is disposed within a targeted zone within a gastrointestinal tract of the subject, so as to increase the Bristol stool score of the fecal matter defecated by the subject.

24. The method of any one of claims 22 to 23, wherein the subject is a subject who has experienced at most one of the following symptoms over the preceding 3 months:
fewer than three bowel movements per week;
straining;
lumpy or hard stools;
sensation of anorectal obstruction;
sensation of incomplete defecation; and
manual maneuvering required to defecate.

25. The method of any one of claims 22 to 23, wherein the subject is a subject who has experienced at most one of the following symptoms over the preceding 3 months:
fewer than three bowel movements per week;
straining during more than 25% of defecations;
lumpy or hard stools in more than 25% of defecations;
sensation of incomplete defecation in more than 25% of defecations;
sensation of anorectal obstruction in more than 25% of defecations; and
manual maneuvering required to facilitate more 25% of defecations.

26. The method of any one of claims 22 to 23, wherein the subject is a constipation free subject.

27. The method of claim 26, wherein said subject suffers from at least one of emotional stress and psychosomatically induced symptoms.

28. The method of any one of claims 1 to 27, said controlling including pre-setting an activation time delay of said capsule, prior to said ingesting.

29. The method of claim 28, wherein the subject is a particular subject and wherein said pre-setting of said activation time delay is according to a measured or estimated transit time of chyme along said gastrointestinal tract of said particular subject.

30. The method of claim 29, further comprising, prior to said pre-setting of said activation time delay, obtaining information relating to said measured or estimated transit time of chime along said gastrointestinal tract of said particular subject.

31. The method of any one of claims 1 to 30, wherein said controlling of said vibrating agitator is effected so as to effect a mechanical stimulation of the wall of said gastrointestinal tract in said targeted zone.

32. The method of any one of claims 1 to 31, wherein said ingesting and controlling together form a treatment session, and wherein said method includes administering to the subject at least one said treatment session.

33. The method of claim 32, wherein said administering to the subject at least one treatment session includes administering to the subject a plurality of treatment sessions.

34. The method of claim 33, wherein said administering a plurality of treatment sessions includes administering at least one said treatment session per week, over a treatment period of at least two weeks, at least at least three weeks, at least four weeks, at least five weeks, at least six weeks, or at least eight weeks.

35. The method of claim 34, wherein said administering at least one treatment session per week includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

36. The method of any one of claims 1 to 35, wherein said targeted zone includes an intestinal section of said gastrointestinal tract of the subject.

37. The method of any one of claims 1 to 35, wherein said targeted zone is the stomach of the subject.

38. The method of any one of claims 1 to 37, wherein said vibrating agitator comprises at least a radial agitation mechanism adapted, in said first vibrating mode of operation, to exert radial forces on said housing, in a radial direction with respect to a longitudinal axis of said housing, thereby to cause said vibrations of said housing.

39. The method of claim 38, wherein said radial agitation mechanism includes unbalanced weight attached to a shaft of an electric motor powered by said battery.

40. The method of any one of claims 1 to 39, wherein said vibrating agitator comprises at least an axial agitation mechanism adapted, in said first vibrating mode of operation, to exert axial forces on said housing, in an axial direction with respect to a longitudinal axis of said housing, thereby to cause said vibrations of said housing.

41. The method of claim 40, wherein said axial agitation mechanism includes an electric motor powered by said battery and an urging mechanism, associated with, and driven by, said electric motor, said urging mechanism adapted to exert said axial forces.

42. The method of claim 41, said urging mechanism adapted to exert said axial forces in opposite directions.

43. The method of claim 41 or claim 42, said urging mechanism adapted to deliver at least a portion of said axial forces in a knocking mode.

44. The method of any one of claims 1 to 37, wherein said vibrating agitator is adapted in said first vibrating mode of operation, to exert radial forces on said housing in a radial direction with respect to a longitudinal axis of said housing and to exert axial forces on said housing in an axial direction with respect to said longitudinal axis of said housing, thereby to cause said vibrations of said housing.

45. The method of claim 44, wherein said vibrating agitator includes a radial agitation mechanism adapted to exert said radial forces and a separate axial agitation mechanism adapted to exert said axial forces.

46. The method of claim 45, wherein said vibrating agitator includes a single agitation mechanism adapted to exert said radial forces and said axial forces.

47. The method of any one of claims 1 to 46, wherein said vibrating mode of operation including a plurality of cycles, each of said cycles including a vibration duration followed by a repose duration, wherein said housing exerts said vibrations during said vibration duration.

48. The method of claim 47, wherein said repose duration is greater than said vibration duration.
